(19) United States
(12) Patent Application Publication
(10) Pub. No.: US 2023/0386682 A1
(43) Pub. Date: Nov. 30, 2023

(54) SYSTEMS AND METHODS TO CHRONOLOGICALLY IMAGE ORTHODONTIC TREATMENT PROGRESS

(71) Applicant: Abdullalbrahim Abdulwaheed, Holbrook, MA (US)

(72) Inventor: Abdullalbrahim Abdulwaheed, Holbrook, MA (US)

(21) Appl. No.: 17/825,489

(22) Filed: May 26, 2022

(51) Int. Cl. G16H 80/00; H04N 5/235; G06V 40/16; G06T 7/00; G06Q 10/10

(52) U.S. Cl. CPC G16H 80/00; H04N 5/2351; G06V 40/16; G06T 7/0016; G06Q 10/1097; G06T 2207/30036

(57) ABSTRACT

A method of chronologically imaging progress of a patient's dental treatment includes providing an executable application to a portable electronic device, the executable application causing a processor to instruct a user to position an image capture device lens towards the user's face, assess ambient lighting condition(s), superimpose an alignment guide on a display screen, instruct the user to align the alignment guide with the user's upper and lower vermillion borders, and capture an image of the user's teeth. A non-transitory computer readable medium and a system to implement the method are also disclosed.

### FIELD OF THE INVENTION

The present invention relates to systems and methods for chronologically imaging ongoing dental treatment or the lack of treatment (relapse). More specifically, to systems and methods for chronologically imaging the progress of a patient's orthodontic treatment and/or condition.

### BACKGROUND

Orthodontics treatment can be used to straighten teeth. Orthodontics treatment can close interstitial gaps between teeth and align teeth to a uniform height. Treatment techniques include clear aligners generated by three-dimensional printers. Computer-aided design systems are used in conjunction with initial intraoral scans or impressions to generate a series of aligners, which incrementally position teeth to achieve a desired result. Similarly, traditional and brackets are also used for orthodontic treatment. Treatment can span months, or even years, depending on the magnitude of the positional changes.

Teeth are always moving. One potential cause of this movement can be that the human alveolar bone is constantly being broken down and built up. As the bone remodels, the teeth can be translocated. A patient undergoing orthodontic treatment can have an interest in monitoring various features as the treatment progresses (e.g., how straight their teeth are, the appearance their smile, etc.). A patient not undergoing treatment can apply embodying systems and/or methods to monitor relapse, or physiological drifting of teeth.

Lacking from the prior art are mechanisms to objectively assess teeth movements by nonprofessionals. In a professional setting, cheek retractors, and very expensive cameras are needed to record those images. Even in a professional setting, there is a need to record one or more images at a consistent distance, and angulation, from the subject during the course of treatment.

### BRIEF DESCRIPTION OF THE DRAWINGS

FIG. 1 is a block diagram of a system in accordance with an embodiment;

FIG. 2 is an environmental view of the manner in which a user begins the process shown in the flowchart depicted in FIG. 3 in accordance with an embodiment;

FIG. 3 is a flowchart depicting a method of capturing an image in accordance with an embodiment;

FIG. 4 is an illustration of an image on a display screen of a handheld device in accordance an embodiment;

FIG. 5 illustrates an alignment guide on a display screen of a handheld device in accordance with an embodiment;

FIG. 6 illustrates the alignment guide of FIG. 5 superimposed on the display screen over a user's facial image in accordance with an embodiment; and

FIG. 7A illustrates a first enhanced alignment guide on a display screen of a handheld device in accordance with an embodiment; and.

FIG. 7B illustrates a second enhanced alignment guide on a display screen of a handheld device in accordance with an embodiment.

### DETAILED DESCRIPTION

Embodying systems and methods obtain images without the need for retractors and/or other dental tools. The user is asked to provide a maximum smile while biting on the back teeth, front teeth or both. Embodiments provide image recording at an about repeatable, consistent distance, angulation and illumination. In accordance with embodiments, a smartphone can be used to obtain a series of chronological images to assess the motion of teeth, alveolar process, and/or soft tissue. Embodiments can include generation of electronic calendar events to alert a user to record images at set intervals.

In accordance with embodiments, these images can be played back as a series of still images, or as a video mashup of the images. Users can objectively determine treatment outcome, relapse, and even the need for treatment. If shared, remote healthcare providers can review one or more of the series of images to adjust/correct treatment, and provide suggestions. Shared images could also be used for promotional or advocacy purpose (Social Media). Such remote review and analysis is important for those undergoing non-, or minimal, supervised treatment.

For purposes of discussion, embodiments applicable to the field of dentistry are disclosed. However, the invention is not so limited. It should be readily understood by persons of ordinary skill in the art the applicability of embodiments to other medical fields.

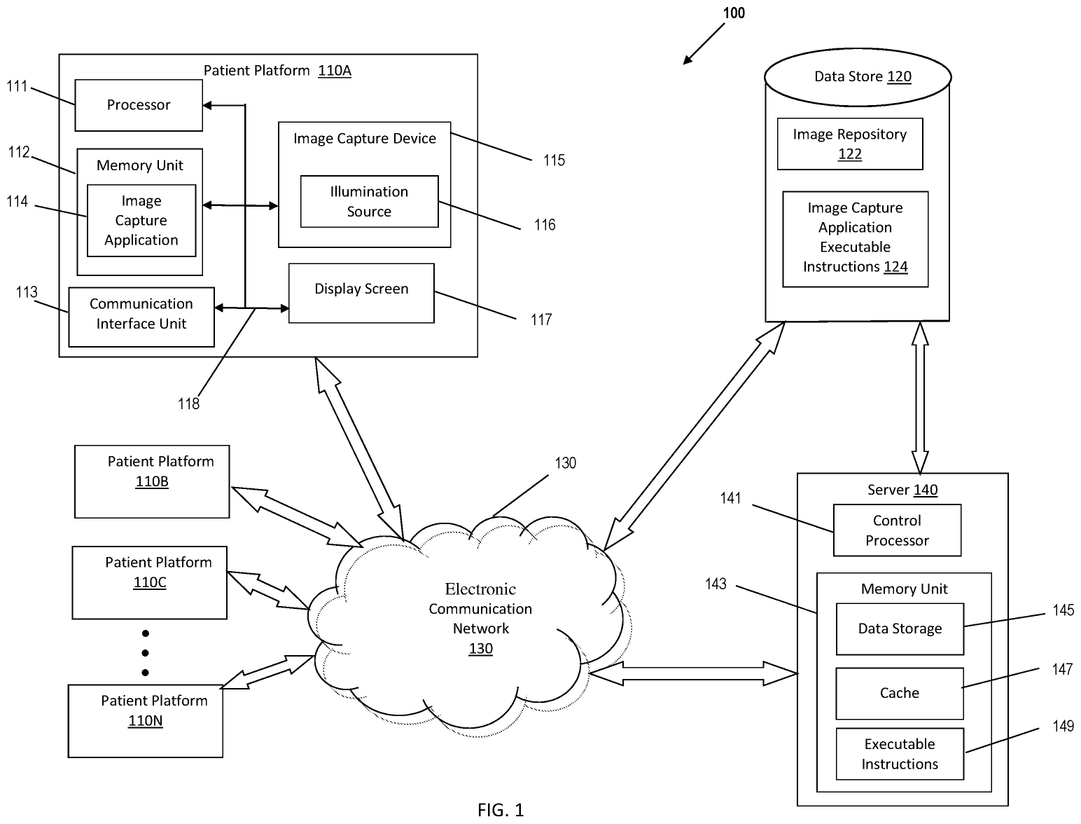

FIG. 1 depicts imaging system 100 in accordance with an embodiment of the invention. System 100 includes patient platform 110A, which can be in communication with electronic communication network 130. This patient platform can be a portable electronic device. Electronic communication network 130 can be the Internet, a local area network, a wide area network, a virtual private network, a wireless area network, a cellular system, or any other suitable configuration of an electronic communication network.

Patient platform 110A can be any type of computing device that includes elements used during obtaining an image—for example, a handheld computing device such as mobile phone, smartphone, tablet computing device, personal digital assistant, etc. In accordance with embodiments, other suitable computing devices can include, but are not limited to, a personal computer, a workstation, a thin client computing device, a netbook, a notebook, etc.

Patient platform 110A includes processor 111 that can access computer readable executable instructions stored in memory unit 112. When executed by the processor, the executable instructions cause the processor to control operations of the patient platform. The processor is in communication with other elements of the patient platform via control/data bus 118.

Communication interface unit 113 conducts, under the control of processor 111, the input/output transmissions of the patient platform 110A. The input/output transmissions can be made by one of several protocols, dependent on the type of computing device. These communication protocols can include, but are not limited to, Ethernet, cellular, Bluetooth, Zigbee, and other communication protocols.

In accordance with embodiments, image capture device 115 can operate in visible, ultraviolet, and/or infrared light spectrums. Still or video images can be captured by the image device. These captured images can be stored in memory unit 112, displayed on display screen 117, and/or communicated to an external device across the electronic communication network 130 via communication interface unit 117.

Illumination source 116 can be used during image capture to illuminate the field-of-view of the image device. In accordance with embodiments, the illumination device can generate illumination of a variety of sizes and intensity in visible, ultraviolet, and/or infrared light spectrums.

In accordance with embodiments, image capture device 115 can operate in visible, ultraviolet, and/or infrared light spectrums. Still or video images can be captured by the image device. These captured images can be stored in memory unit 112, displayed on display screen 117, and/or communicated to an external device across the electronic communication network 130 via communication interface unit 117.

Illumination source 116 can be used during image capture to illuminate the field-of-view of the image device. In accordance with embodiments, the illumination device can generate illumination in visible, ultraviolet, and/or infrared light spectrums.

Image capture application 114 is a set of executable instructions located in memory unit 112, which when executed cause the patient platform to be used as a remote evaluation and diagnostic tool by the practitioner. The image capture evaluation application can be an application file pre-installed on the patient platform or obtained as a downloadable file from an application repository.

Implementations of the invention are not limited to a single patient platform 110A. It is readily understood that the present disclosure can support multiple patient platforms 110A, 110B, 110C, . . . , 110N. Each patient platform can correspond to an individual user.

Data store 120 can include image repository 122 that contains one or more images, or series of images. In implementation, there can be more than one user obtaining images on respective patient platforms 110A, 110B, 110C, . . . , 110N. Each user's image(s) can be indexed within the image repository for access by just that user, and a designated practitioner to maintain privacy and confidentiality.

Server 140 is in communication across the electronic communication network 130 with the patient platforms 110A, 110B, 110C, . . . , 110N. The server is also in communication with data store 120. The server can include a control processor 141, a memory unit 143, and communication interface(s) (not shown). The memory unit can include data storage 145, cache 147, and executable instructions 149.

Server 140 performs database management services including access images on each patient platform 110A, 110B, 110C, . . . , 110N and index the images' aggregation into the data store. Executable instructions 149 can cause control processor 141 to implement the image aggregation into the data store, read the stored data, transfer the image capture application 124 to individual patient platforms.

Embodiments can be implemented as a computer-based application (“app”), as may be utilized by smartphones or similar portable computing devices equipped with a camera. An embodying image capture application provides guidance in the form of direction instructions for the user and control of the illumination source.

Because facial anatomy includes several amorphous angulations, with less than distinct margins. Inconsistent ambient lighting, distance from the image capture device 115 to the user, orientation of the image capture device in reference to the user, and other variables all act to introduce inconsistent positioning of the camera for each image of the chronological series. Embodying techniques overcome, and/or minimize, these and other positioning/lighting variables to provide consistent images over time. Embodiments provide lighting instructions, a lighting source, detecting image capture device angulation, and (as-needed) providing positional correction instruction, an image acquisition mechanism, and image acquisition instructions in a mobile solution for capturing consistently-positioned images over one or more intervals of time. The acquisition instructions can be provided via audio, video, or a combination of audio-video.

The method utilizes operations to standardize various aspects of the image detection and capture process including standardizing the level of ambient lighting during image detection, establishing a set distance and angulation of the camera relative to the user, providing mechanisms to clearly outline and differentiate the desired facial anatomy (teeth) of interest from adjacent areas. The captured image of the user's teeth can then be stored locally on patient platform 110A. These captured images can also be provided to data store 120 for storage in image repository 122.

Embodying methods include steps to create a controlled environment for capturing an image by a user at an about consistent, and repeatable, position in front of the user's mouth. An embodying method provides control of ambient lighting and camera distance, angulation, from a user in order to create a reproducible environment for image capture. To create a reproducible environment for image capture, embodying methods can monitor distance, and angulation from soft-tissue facial landmarks of a user in relation to a custom silhouette.

In accordance with embodiments, the image capture application 114 guides the user through a process that allows the user to obtain an about consistent and repeatable-positioned images of their teeth. The app 114 can provide instructions to the user through the display screen 117.

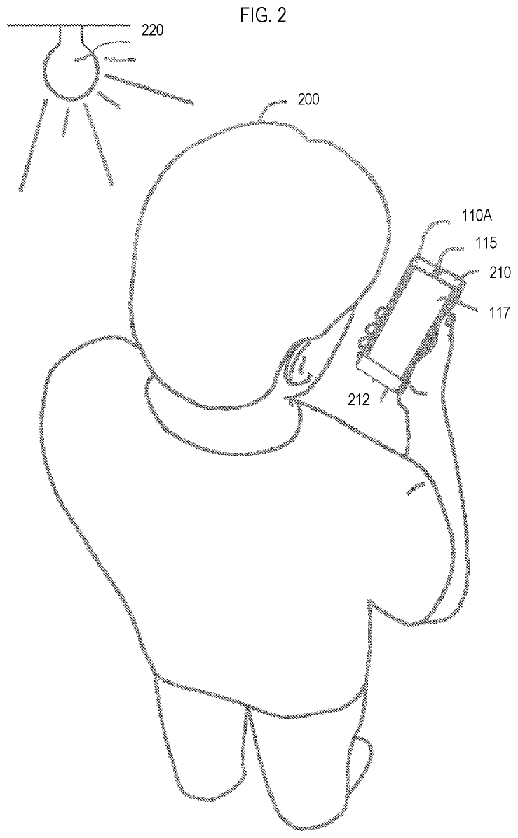

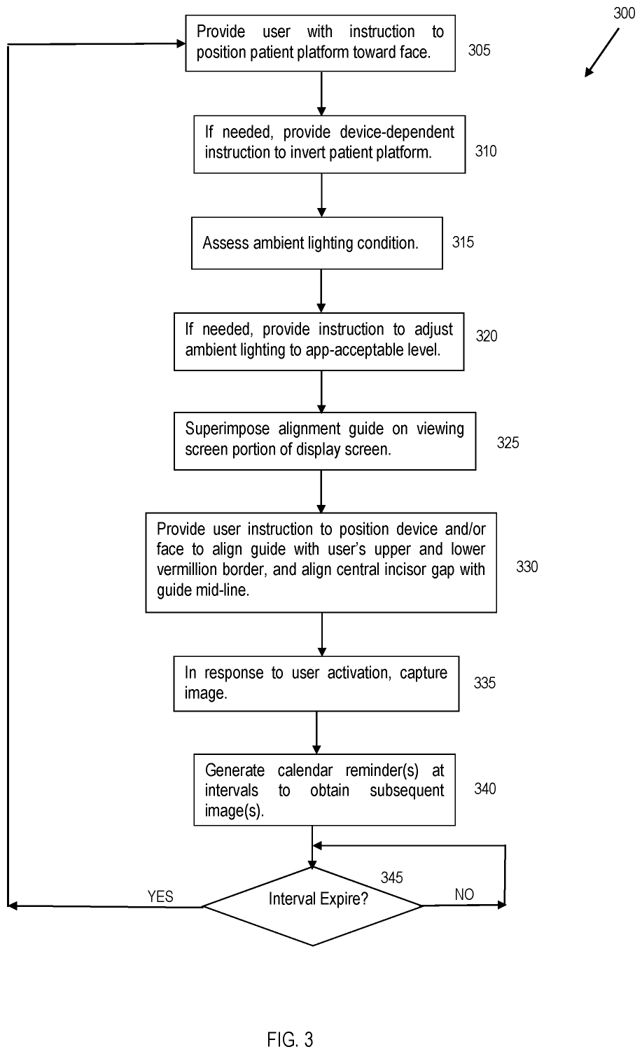

FIG. 2 is an environmental view of the manner in which a user 200 begins an embodying process shown in the flowchart depicted in FIG. 3. The method begins with the user 200 holding the patient platform 110A in front of their face so that the front-facing (user-side) image capture device 115 is able to detect and display a real time image of the user's face on the display screen 117.

FIG. 3 is a flowchart of process 300 for capturing mages of orthodontic treatment from a repeatable position in accordance with an embodiment. The app 114 provides the user with instruction to position the patient platform toward their face, step 305. An instruction to invert the patient platform is provided, step 310. This instruction to invert the device is given if the device has its forward-facing image capture lens at the top of the device (i.e., inversion is a device-dependent instruction). By inverting the device, the image capture lens is more closely positioned across from the user's mouth.

Ambient lighting conditions are assessed by the app 114, step 315. The app can provide instruction to adjust the ambient light source(s) to an acceptable level, step 320. The app can access information from the patient platform's light sensors to determine the level of ambient lighting. The primary source of lighting the image subject area is produced by a residual portion 422 of the display screen 117. This primary source of lighting is controlled by the app based on ambient lighting conditions.

An alignment guide is superimposed on the viewing portion 420 of the display screen, step 325. The app provides the user with instruction to position the device and/or their face to align the guide with the user's upper and lower vermillion borders, and align the central incisor gap with the displayed vertical midline of the guide, step 330. In implementation, alignment can also include instruction to align the center of the patient's face (e.g., center of the nose, or the philtrum). In accordance with an embodiment, other soft tissue landmarks can be used to align the face and image capture device.

Step 330 can be implemented in more than one action. Video instruction can be provided on the display screen to facilitate this alignment. In an embodiment, the user can be instructed to record images from two different physiological configurations. A first position where the front teeth are in contact only; and a second position where the back teeth are in contact.

The user determines satisfactory alignment of their mouth (and other soft tissue landmarks) to the guide, and activates the image capture device, step 335. The app can generate, step 340, calendar reminders to obtain subsequent images at set intervals, step 340. After determining an interval has expired, step 345, the app returns to step 305. Captured images can be stored local to the patient platform and/or remotely in a data store.

To further enhance the alignment guide, the user can trace a custom soft tissue guide on the display screen after recording a first image. The custom alignment guide can be used for users with greater anatomical variations. In another iteration, the user may dismember the existing silhouettes into its components, and utilizes the fragments in constructing a custom guide.

If the image capture device includes a proximity sensor, in accordance with an embodiment, this incorporated proximity sensor can be used to help guide the user in aligning the silhouette.

For purposes of discussion, the following implementation uses a smartphone as the patient platform 110A and uses a camera and lens as the image capture device 115. The smartphone 110A has a top 210 and a bottom 212 with such relative terms being applied from the perspective of the user.

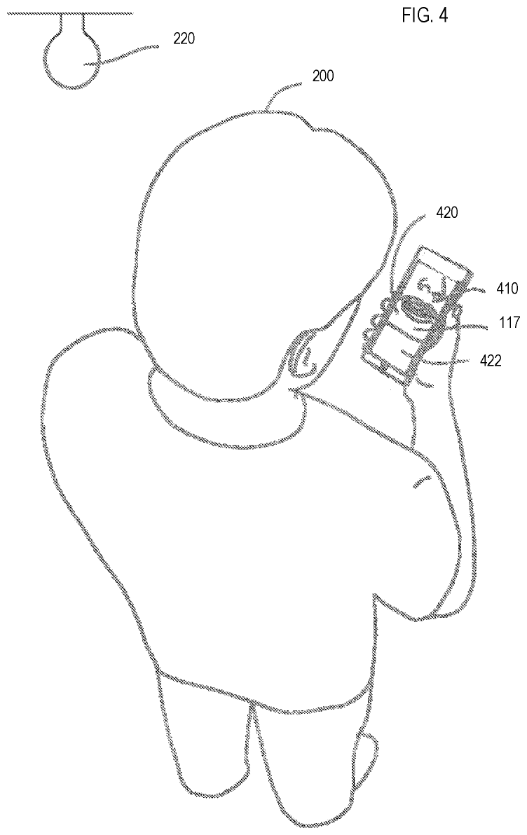

FIG. 4 illustrates a user's facial image 410 on a display screen of a handheld device in accordance with embodiments. When obtaining the facial image 410, the smartphone 110A creates two regions on the display 117. The first region is a viewing screen 420 that shows the user's facial image 410; and the second region is a residual screen 422 used as a light emission source. The allocation of the display 117 into these two regions can be varied dependent on ambient lighting, sizing of the facial image, and other factors and/or considerations. The residual screen 422 is illuminated to emit lighting of a specific color temperature. By varying the RGB values and pixel emission intensity of individual pixels a variety of source light temperatures are possible.

In some embodiments the application is in communication with an internal gyroscope or other orientation-sensing mechanism of the smartphone so as to measure the tilt of the smartphone 110A. When the user's facial image 410 displayed on the viewing screen 422 meets positional constraints, the app 114 can record the tilt, roll, and yaw positional data for the device upon initial image capture/calibration. In accordance with embodiments, this recorded positional data can be used for subsequent image captures to ensure that the user's facial image 410 is captured by the camera from about a consistent and repeatable position of the smartphone with respect to the user.

Subsequent images can be obtained at regular and/or irregular temporal intervals, with some intervals extending into weeks or months after the initial image capture. By capturing the series of images from about a repeatable position with respect to the image subject, the effect of parallax can be removed, or minimized, to have a negligible impact on each of the images in the series. Embodying methods align the camera-to-subject to remove, or minimize, the positional displacement of the camera-to-subject that can be introduced for each image capture. This alignment can position the camera in all six degrees of freedom (i.e., x-, y-, and z-planes; roll, pitch, and yaw) to the about repeatable position of the former images captured in the series.

As part of the calibration process, the app 114 can provide an audio and/or visual prompt to appear on the screen 117 for the user to invert the smartphone 110A “upside down” or to “rotate the smartphone 180 degrees”, etc. so that the camera 115 is now positioned at the lower end of the smartphone from the perspective of the user 200. In this position the camera 115 is better positioned to detect and display an image of the user's face on at least a portion of the screen 117. Note that the user's face can appear correctly orientated on the display screen 117 despite the camera 115 being inverted.

The app can also prompt the user 200 to “turn off all the lights in a room” or to “go into a darkened room”, etc. Instead of relying on consistent ambient lighting for each image, embodiments use the light emitted from residual screen 422 to illuminate the user 200.

Reliance on the residual screen for lighting provides consistent lighting conditions for obtaining each image of the user's face 410.

In some embodiments, the smartphone 110A can have one or more light sensors. If the ambient illumination (extraneous lighting captured by the camera; e.g., from light source 220 or other environmental light sources), exceeds an app-determined limit, the app can place a prompt on the display screen 117 (and/or provide an audio indication) that the light level is too bright for acceptable calibration. The calibration process can be placed on hold until the ambient light level achieves the required threshold.

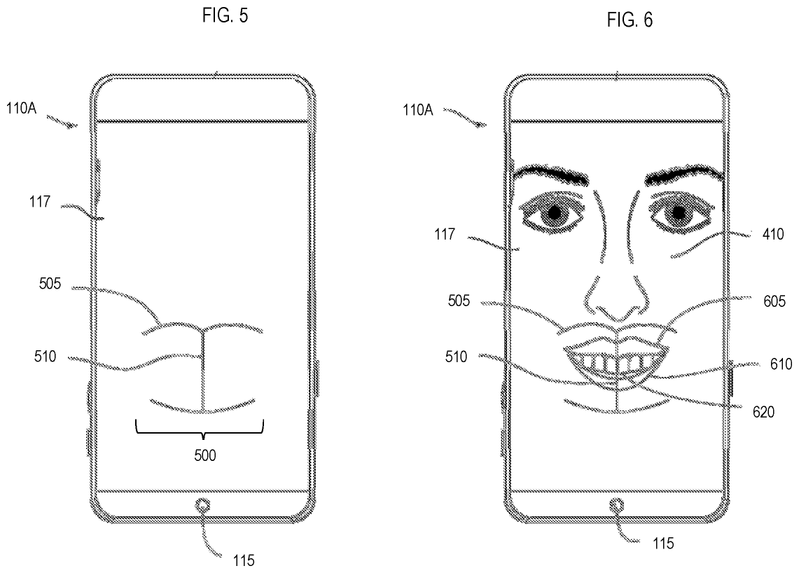

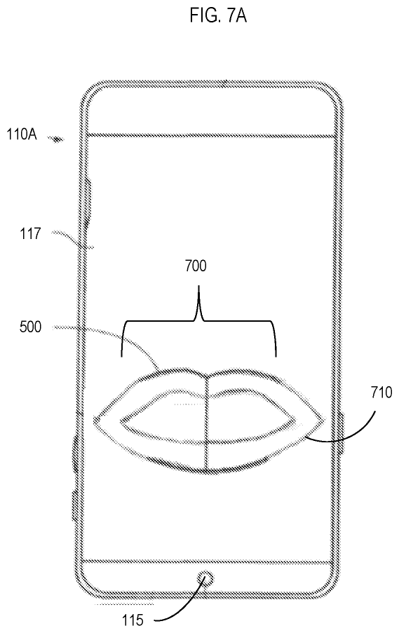

FIG. 5 illustrates an alignment guide 500 on a display screen 117 of an image capture device 110A in accordance with an embodiment. FIG. 6 illustrates the alignment guide of FIG. 5 superimposed on the display screen over a user's facial image 410 in accordance with an embodiment. FIG. 6 illustrates the alignment guide not yet aligned with the user's upper and lower vermillion borders.

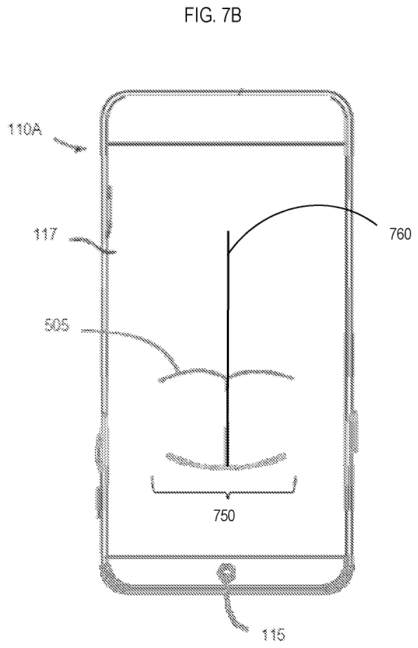

FIG. 7A illustrates a first enhanced alignment guide 700 on display screen 117 of a smartphone in accordance with an embodiment. The first enhanced alignment guide 700 includes alignment guide 500 and a graphical representation of a person's lips 710. This graphical representation 710 can have a transparent, or translucent, fill. FIG. 7B illustrates a second enhanced alignment guide 750 on display screen 117 of a smartphone in accordance with an embodiment. The second enhanced alignment guide 750 includes alignment guide 500 with an extended vertical midline 760. In some implementations, this extended vertical midline can be used as a guide for the user to better position the image capture device with the center of their face. Alignment of the extended vertical line 760 can be with the philtrum or the middle of the nose.

In accordance with embodiments, to position the camera 115 at about a repeatable position with respect to the target image (e.g., the face of user 200), the user can position his or herself in such a manner so as to align his/her upper vermillion border 605 and lower vermillion border 610 (i.e., lips) within a portion of the display screen outlined by the alignment guide 500. The application may prompt the user to “smile” before or during this step. In some embodiments, the upper vermillion border may consist of a specific anatomical shape contiguous with the philtrum (i.e., the region between the nose and upper lip of the user). The alignment guide can also include a custom drawn vermillion border, after registration of the initial image.

In addition to aligning the upper vermillion border 605 and lower vermillion border 610 within the alignment guide 500, while the user is smiling or otherwise exposing at least some of teeth, in an embodiment the user 200 can also align the philtrum or the middle or the nose, or face with alignment guide. In an embodiment that uses the enhanced alignment guide, the graphical representation of lips 710 further assists the user to align their face to the image capture device at an about repeatable position. Embodiments are not so limited to achieve alignment with the philtrum or vermillion border. In other implementations, other soft tissue landmarks can be used individually or in combination—for example, the nose tip with the lip commissures.

Once the lips and midline are properly aligned and in place within the superimposed graphics of the alignment guide 500 (or the enhanced alignment guide 700), the user may activate the camera to capture the image of the user's face 410. In accordance with an embodiment, the image 410 may also be automatically recorded utilizing facial recognition technology. The distance between the upper and lower vermillion border, along with the midline and its angulation is fixed as is the distance between the user and the device.

Using the alignment guide (e.g., alignment guide 500 or enhanced alignment guide 700) utilizes the upper and lower lip, midline of the nose or philtrum, for camera-to-subject positioning at an about repeatable location between image captures. In accordance with embodiments, the camera-to-subject distance is about constant between image captures. This approach also provides an about consistent angulation/orientation of the camera. During most orthodontic treatment, minor soft tissue changes occur. Using the alignment guide ensures that the images are captured at an about repeatable and consistent position that uses landmarks external to the mouth; and is not dependent on soft tissue facial features, thus comparison between images results in an accurate determination of teeth movement.

The soft tissue features of adults rarely change with orthodontics. However, with children their soft tissue features do change. In accordance with embodiments, to properly align children to the silhouette in addition to their lips, other soft tissue landmarks can be used. These soft tissue landmarks can include, but are not limited to, the eyes, the forehead, the tip of the nose, the zygoma (cheekbone), and the ears.

In accordance with embodiments, the app can generate electronic calendar events to alert a user to capture a new image of their teeth. The alert interval(s) can be selected by the user based on the user's treatment status. For example, if the user is in active orthodontic band-and-bracket treatment images could be obtained after each adjustment occurs; if the user is undergoing clear aligner treatment, a typical interval to register an image could be every two weeks. Alternatively, the user can set the interval to register new images at their own preferred interval. These intervals need not be uniform in temporal distance.

In accordance with embodiments, these images can be played back as a series of still images, or as a video mashup of the images. Users can objectively determine treatment outcome, relapse, and even the need for treatment. If shared, remote healthcare providers can review one or more of the series of images to adjust/correct treatment, and provide suggestions. Such remote review and analysis is important for those undergoing non-, or minimal, supervised treatment.

In accordance with embodiments, the app can estimate the spacing, or lack thereof, in the user's dental dimension by applying known dimensions of the brackets, or orthodontic buttons, or a calibration target, to the known distance and angulation between the user's face and imaging device. This spacing estimate can be useful in providing a remote clinician with treatment-related information. This technique can be used to measure hard and soft tissue anatomy, and pathology.

In accordance with embodiments, captured images can be stored locally to the patient platform. Additionally, the captured images can be stored in the image repository 122 of data store 120 across the electronic communication network 130. Server 140 can coordinate the storage and retrieval of the stored captured images. The server includes executable instructions that can cause control processor 141 to index the captured images by user (i.e., for multiple users), and to retrieve the captured images.

These stored captured images can be accessed by the user, and/or their dental professional, to playback as a series of still images to form a video of treatment progress. Individual images can be retrieved from the image repository 122 to evaluate treatment progress across a temporal interval (e.g., weeks, months, etc.) to assess progress.

In accordance with an embodiment, a treating orthodontist can retrieve a user's images to evaluate progress during treatment. This feature provides an objective method to supervise a distant patient.

In another implementation, for individuals not yet under orthodontic treatment (or for individuals that are experiencing post-treatment relapse), the still images can be used individually, and/or collectively to form a video, as an aide to visualize future treatment recommendations.

In accordance with embodiments, and edge detection image analysis algorithm can be applied to the captured images. The edge detection algorithm can define the edges of individual teeth. The user and/or orthodontist can then isolate the image of a single tooth and track its motion over time. Edge detection results can be used (manually or via computerized analysis) to identify teeth that are improperly shifting position. By comparing edge detection results on successive images, the analysis can identify teeth that are diverging from, or impinging on, neighboring teeth. If the user enters a single standard measurement, including a width of a tooth, software can provide the orthodontist and the user with anatomical dimensions that could aid in treatment.

In accordance with an embodiment of the invention, a computer program application stored in non-volatile memory or computer-readable medium (e.g., register memory, processor cache, RAM, ROM, hard drive, flash memory, CD ROM, magnetic media, etc.) may include code or executable computer instructions 62 that when executed may instruct or cause a controller or processor to perform methods discussed herein such as a method for capturing from an about repeatable position a chronological series of images of ongoing orthodontic treatment to evaluate progress.

The computer-readable medium may be a non-transitory computer-readable media including all forms and types of memory and all computer-readable media except for a transitory, propagating signal. In one implementation, the non-volatile memory or computer-readable medium may be external memory.

Although specific hardware and data configurations have been described herein, note that any number of other configurations may be provided in accordance with embodiments of the invention. Thus, while there have been shown, described, and pointed out fundamental novel features of the invention as applied to several embodiments, it will be understood that various omissions, substitutions, and changes in the form and details of the illustrated embodiments, and in their operation, may be made by those skilled in the art without departing from the spirit and scope of the invention. Substitutions of elements from one embodiment to another are also fully intended and contemplated. The invention is defined solely with regard to the claims appended hereto, and equivalents of the recitations therein.

In accordance with embodiments, one or more captured images can be analyzed for the propose of recommending patient-appropriate orthodontic treatment types. Analysis of the captured images can be performed by remote clinicians in determining if the remote clinician is qualified to provide treatment.

Abnormal teeth movement, alveolar process change, can be indicative of abnormalities in bone metabolism. Or might not be related to endocrine or bone related bone pathology. Embodying systems and methods can be used in detecting and/or monitoring pathologies, and in the prediction of treatment outcome.

1. A method of chronologically imaging progress of a patient's dental treatment, the method comprising:
providing an executable application to a portable electronic device, the portable electronic device including a control processor, an image capture device, and a display screen;
the executable application causing the control processor to perform the method, the method including:
providing the user with instruction to position a lens of the image capture device towards the user's face;
assessing ambient lighting condition for suitability in capturing an image;
superimposing an alignment guide on the display screen;
providing the user with instruction to position the alignment guide with the user's upper and lower vermillion borders; and
capturing an image of the user's teeth.

2. The method of claim 1, based on the image capture device lens being located at an upper end of the portable electronic device, the method including providing instruction to the user to invert the portable electronic device.

3. The method of claim 1, based on an assessment that the ambient lighting condition is not suitable for capturing an image, the method including providing instruction to the user to adjust the ambient lighting condition.

4. The method of claim 1, including instructing the user to align a vertical midline of the alignment guide with one of their central incisor gap, philtrum center, or facial midline.

5. The method of claim 1, including generating at least one calendar event reminder to capture a subsequent image of the user's teeth.

6. The method of claim 5, the at least one calendar reminder occurring at an interval selected by the user or a dental professional.

7. The method of claim 5, based on an occurrence of the calendar reminder, including notifying the user to obtain a subsequent image of the user's teeth.

8. The method of claim 1, the alignment guide including an outline of an upper vermillion border, a lower vermillion border, and a vertical midline.

9. The method of claim 8, the alignment guide including a graphical representation of lips.

10. The method of claim 1, prior to capturing the image of the user's teeth, including one of:
receiving an indication from the user that alignment is complete; and
using a facial recognition technique to determine that alignment is complete.

11. The method of claim 10, including after the alignment is complete, obtaining subsequent images of the user's teeth from an about repeatable distance and angulation by using a same alignment guide as used for an initial captured image of the user's teeth.

12. The method of claim 1, including:
generating a playback image having a series of captured images of the user's teeth; and
determining if a relapse is occurring or if an adjustment to treatment is needed by reviewing the playback image.

13. The method of claim 12, based on the determination, developing a treatment plan or a revised treatment plan.

14. A non-transitory computer readable medium having stored thereon instructions which when executed by a processor cause the processor to perform a method of chronologically imaging progress of a patient's dental treatment, the method comprising:
providing an executable application to a portable electronic device, the portable electronic device including a control processor, an image capture device, and a display screen;
the executable application causing the control processor to perform the method, the method including:
providing the user with instruction to position a lens of the image capture device towards the user's face;
assessing ambient lighting condition for suitability in capturing an image;
superimposing an alignment guide on the display screen;
providing the user with instruction to position the alignment guide with the user's upper and lower vermillion borders; and
capturing an image of the user's teeth.

15. The computer readable medium of claim 14, based on the image capture device lens being located at an upper end of the portable electronic device, including executable instructions to cause the processor to perform the method, including providing instruction to the user to invert the portable electronic device.

16. The computer readable medium of claim 14, based on an assessment that the ambient lighting condition is not suitable for capturing an image, including executable instructions to cause the processor to perform the method, including providing instruction to the user to adjust the ambient lighting condition.

17. The computer readable medium of claim 14, including executable instructions to cause the processor to perform the method, instructing the user to align a vertical midline of the alignment guide with one of their central incisor gap, philtrum center, or facial midline.

18. The computer readable medium of claim 14, including executable instructions to cause the processor to perform the method, including generating at least one calendar event reminder to capture a subsequent image of the user's teeth.

19. The computer readable medium of claim 18, including executable instructions to cause the processor to perform the method, including the at least one calendar event reminder occurring at an interval selected by one of the user and a dental professional.

20. The computer readable medium of claim 18, based on an occurrence of the calendar reminder, including executable instructions to cause the processor to perform the method, including notifying the user to obtain a subsequent image of the user's teeth.

21. The computer readable medium of claim 14, the alignment guide including an outline of an upper vermillion border, a lower vermillion border, and a vertical midline.

22. The computer readable medium of claim 21, the alignment guide including a graphical representation of lips.

23. The computer readable medium of claim 14, including executable instructions to cause the processor to perform the method, prior to capturing the image of the user's teeth, the method including one of:
receiving an indication from the user that alignment is complete; and
using a facial recognition technique to determine that alignment is complete.

24. The computer readable medium of claim 23, including executable instructions to cause the processor to perform the method, including after the alignment is complete, obtaining subsequent images of the user's teeth from an about repeatable distance and angulation by using a same alignment guide as used for an initial captured image of the user's teeth.

25. The computer readable medium of claim 14, including executable instructions to cause the processor to perform the method, including executable instructions to cause the processor to perform the method, including:
generating a playback image having a series of captured images of the user's teeth; and
determining if a relapse is occurring or if an adjustment to treatment is needed by reviewing the playback image.

26. The method of claim 25, based on the determination, developing a treatment plan or a revised treatment plan.
